(19) United States
(12) Patent Application Publication
(10) Pub. No.: US 2003/0032895 A1
(43) Pub. Date: Feb. 13, 2003

(54) Dual action aspiration biopsy needle

(76) Inventor: John S. Fisher, Belleair, FL (US)

Correspondence Address: SMITH & HOPEN PA, 15950 BAY VISTA DRIVE, SUITE 220, CLEARWATER, FL, 33760

(21) Appl. No.: 09/682,252

(22) Filed: Aug. 9, 2001

(51) Int. Cl. A61B 5/00

(52) U.S. Cl. 600/573; 600/576

(57) ABSTRACT

A dual action biopsy needle collects tissue from a lesion during forward and rearward reciprocations of the needle along its longitudinal axis of symmetry. A first cutting edge, formed by a beveled distal end of the needle, slices tissue during proximal-to-distal travel of the needle. A second cutting edge is provided by a transversely disposed slot formed in the needle near the first cutting edge. The second cutting edge slices tissue from a lesion during distal-to-proximal travel of the needle. In a first embodiment, the second cutting edge is coincident with an exterior surface of the needle. In a second embodiment, the second cutting edge is elevated with respect to the exterior surface and in a third embodiment the second cutting edge is recessed. Additional embodiments include a second slot, a channel, and a hinge for enabling pivotal movement of the second and third cutting edges.

### BACKGROUND OF INVENTION

1. Field of the Invention

This invention relates, generally, to aspiration biopsy needles. More particularly, it relates to an aspiration biopsy needle having an enhanced cellular material collection capability.

2. Description of the Prior Art

A biopsy procedure collects cellular material from a suspect lesion in soft tissue such as breast tissue for analysis in a laboratory to determine whether or not the lesion is malignant. Biopsy needles are used to collect samples of the lesion because such needles are less invasive than conventional surgery and thus minimize trauma and reduce or eliminate recovery time. Biopsy needles collect samples by aspiration; a vacuum applied to the proximal end of a hollow needle pulls severed cellular material from the lesion into the lumen of the needle. The needle is then retracted from the soft tissue so that the cellular material in the lumen can be removed for analysis. If more samples of the lesion are needed, the biopsy needle is reintroduced into the soft tissue of the patient.

Sometimes, however, the biopsy procedure fails to collect a sample of sufficient size to enable laboratory analysis. When that happens, the physician must repeat the procedure, causing additional trauma to the breast or other body part undergoing biopsy.

Although conventional aspiration biopsy needles represent a significant improvement over more invasive procedures for performing biopsies on suspect lesions, there remains a need for an improved biopsy procedure that increases the amount of sample that may be recovered with one needle insertion so that multiple insertions need not be performed to complete a biopsy procedure.

However, in view of the prior art considered as a whole at the time the present invention was made, it was not obvious to those of ordinary skill in the pertinent art how the well-known biopsy needle could be improved.

### SUMMARY OF INVENTION

The longstanding but heretofore unfulfilled need for a biopsy needle having enhanced cellular material collection capability is now met by a new, useful, and nonobvious invention. The novel aspiration biopsy needle includes a needle of elongate, hollow construction having a proximal end and a beveled distal end. The beveled distal end includes a first cutting edge that slices tissue when the needle is inserted into a patient, said needle being displaced from a proximal position to a distal position during insertion. The needle has a uniform diameter along a substantial part of its extent. A slot is formed in the needle near the beveled distal end; the slot is transversely disposed relative to a longitudinal axis of the needle. The slot is also angled relative to a transverse axis of the needle such that a bottom of the slot is positioned distal to an opening of the slot. The opening of the slot includes a second cutting edge that slices tissue when the needle is displaced from a distal position to a proximal position. The novel structure further includes conventional means for applying a vacuum to a proximal end of the needle so that cellular material sliced by the first cutting edge during proximal-to-distal travel of the needle is pulled into a lumen of the needle and so that cellular material sliced by the second cutting edge during distal-to-proximal travel of the needle is also pulled into the lumen.

In all embodiments, the slot has a circumferential extent of about one half the circumference of the needle.

In a first embodiment, the second cutting edge is coincident with the exterior surface of the needle.

In a second embodiment, the second cutting edge is raised with respect to the exterior surface of said needle, and in a third embodiment, the cutting edge is recessed with respect to said exterior surface.

In a fourth embodiment, the slot is also angled relative to a transverse axis of the needle such that a bottom of the slot is positioned proximal to an opening of the slot and the second cutting edge thereby created is coincident with the exterior surface if the needle.

In fifth and sixth embodiments, the second cutting edge of the fourth embodiment is elevated and recessed, respectively, relative to the exterior surface of the needle.

In a seventh embodiment, a first slot is formed in the needle as in the first embodiment and a second slot is formed in the needle as in the fourth embodiment. The first and second slots are longitudinally spaced apart from one another.

In an eighth embodiment, a transversely disposed channel is formed in the needle and provides a second and a third cutting edge that are coincident with the exterior surface of the needle.

In ninth and tenth embodiments, the second and third cutting edges, respectively, are elevated with respect to the exterior surface of the needle and in eleventh and twelfth embodiments, the second and third cutting edges, respectively, are recessed with respect to said exterior surface.

In additional embodiments, the second cutting edge is mounted for pivotal movement about a transversely disposed hinge. The hinge enables the second cutting edge to open wider relative to its non-hinged position so that it may cut thicker slices of tissue as the needle is displaced in a distal-to-proximal direction.

In still further embodiments, the third cutting edge is mounted for pivotal movement about a transversely disposed hinge and in additional embodiments, both the second and third cutting edges are so mounted.

A primary object of the invention is to provide an aspiration biopsy needle that collects a greater quantity of cellular material per needle insertion than conventional aspiration biopsy needles.

A closely related object is to accomplish the foregoing object by making a structural change to an existing biopsy needle so that physicians will have a sense of familiarity when employing the improved needle.

These and other important objects, advantages, and features of the invention will become clear as this description proceeds.

The invention accordingly comprises the features of construction, combination of elements, and arrangement of parts that will be exemplified in the description set forth hereinafter and the scope of the invention will be indicated in the claims.

### BRIEF DESCRIPTION OF DRAWINGS

For a fuller understanding of the nature and objects of the invention, reference should be made to the following detailed description, taken in connection with the accompanying drawings, in which:

FIG. 1 is a perspective view of a first embodiment of the novel aspiration biopsy needle;

FIG. 2 is an enlarged perspective view of the distal end of the needle of said first embodiment;

FIG. 3 is an enlarged longitudinal sectional view of the parts depicted in FIG. 2;

FIG. 4 is an enlarged perspective view of the distal end of a second embodiment;

FIG. 5 is an enlarged longitudinal sectional view of the parts depicted in FIG. 4;

FIG. 6 in an enlarged longitudinal sectional view of a third embodiment;

FIG. 7 is an enlarged longitudinal sectional view of a fourth embodiment;

FIG. 8 is an enlarged longitudinal sectional view of a fifth embodiment;

FIG. 9 is an enlarged longitudinal sectional view of a sixth embodiment;

FIG. 10 is an enlarged longitudinal sectional view of a seventh embodiment;

FIG. 11 is an enlarged longitudinal sectional view of a eighth embodiment;

FIG. 12 is an enlarged sectional view of a ninth embodiment;

FIG. 13 is an enlarged sectional view of a tenth embodiment;

FIG. 14 is an enlarged sectional view of an eleventh embodiment;

FIG. 15 is an enlarged sectional view of a twelfth embodiment; and

FIG. 16 is an enlarged sectional view of a thirteenth embodiment.

### DETAILED DESCRIPTION

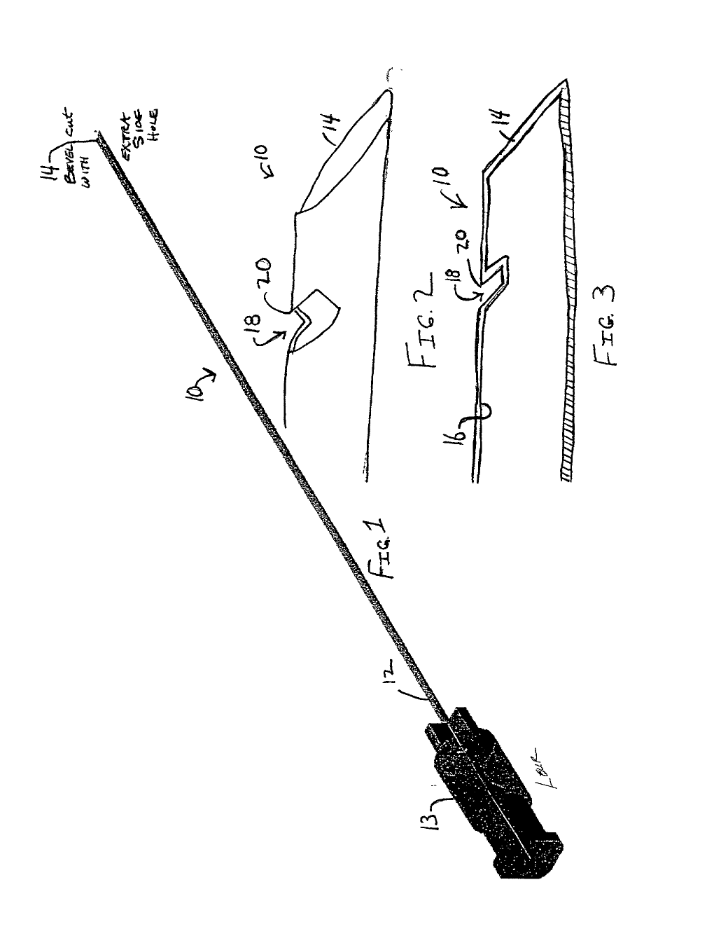

Referring now to FIGS. 1-3, it will there be seen that the reference numeral 10 denotes a first illustrative embodiment of the present invention.

The proximal end 12 of aspiration biopsy needle 10 is mounted in a LUER LOK® adapter 13 or equivalent structure to which a vacuum is applied from a remote source of negative pressure, not shown, when the leading or distal end 14 of said needle is inserted into a suspect lesion in soft tissue. The sharp leading end 14 is beveled in a well-known way to form a first cutting edge that cuts or slices the lesion as it travels therethrough in a proximal-to-distal direction.

A vacuum applied to adapter 13 pulls cellular material severed from the lesion into lumen 16 of the needle. The well-known prior art procedure ends at this point, i.e., the needle having a sample of the lesion in its lumen is retracted from the soft tissue and the sample is delivered to a lab for analysis. Additional sample-taking requires additional insertions of the needle to the site of the lesion.

A first innovation of this invention includes slot 18 formed in needle 10, near first cutting edge 14 thereof. Slot 18 is transversely disposed relative to a longitudinal axis of the needle. A lower end or bottom of the slot is distal to the open upper end thereof. A second cutting edge 20 is formed at the distal edge of the upper end of the slot. Accordingly, second cutting edge 20 slices cellular material from a lesion when the needle is retracted, i.e., when the needle is displaced along its longitudinal axis in a distal-to-proximal direction.

It should therefore be understood that a physician may reciprocate needle 10 along its longitudinal axis, collecting slices of a suspect lesion that are cut by the first cutting edge 14 during the proximal-to-distal portion of each reciprocation, and collecting slices of said lesion that are cut by second cutting edge 20 during the distal-to-proximal portion of each reciprocation. Thus, the chances of collecting a quantity of lesion that is sufficient for analysis is greatly enhanced by the provision of second cutting edge 20.

The circumferential extent of slot 18 is approximately equal to half of the circumference of needle 10, but that structural feature of slot 18 is not critical to this invention; the circumferential extent of said slot could be more or less than that described and depicted.

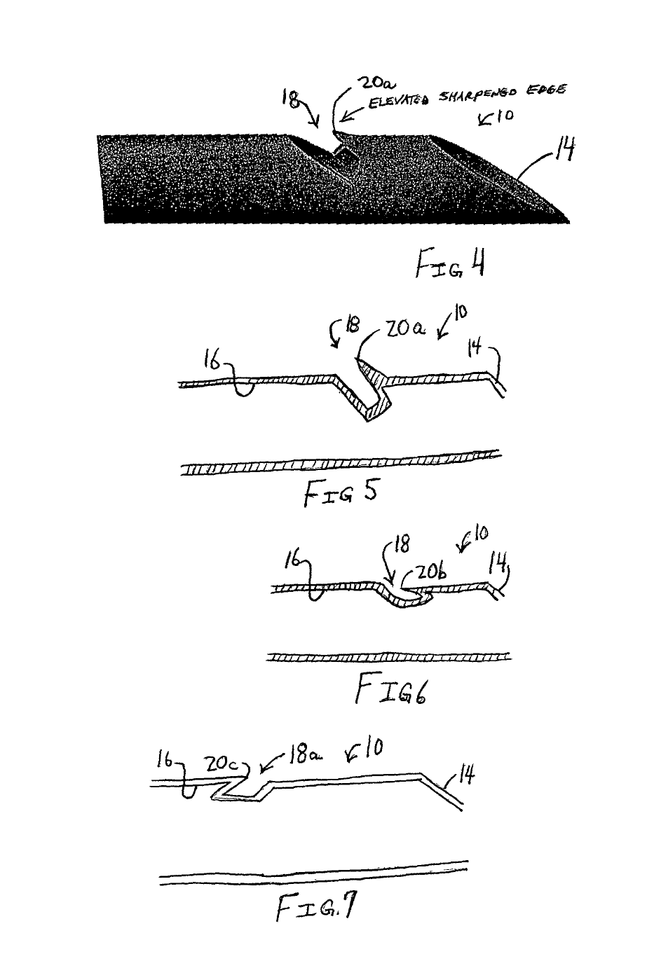

As perhaps best depicted in FIG. 3, second cutting edge 20 is coincident or flush with the exterior surface of needle 10 in this first embodiment FIG. 4 provides a perspective view of a second embodiment of needle 10 where second cutting edge 20a is elevated with respect to the exterior surface of needle 10 and FIG. 5 provides a longitudinal sectional view of said second embodiment. The protrusion of second cutting edge 20a above the exterior surface of the needle ensures that the cellular material collected when using this second embodiment should be greater than the amount of cellular material collected when using the first embodiment.

FIG. 6 provides a longitudinal sectional view of a third embodiment where second cutting edge 20b is recessed with respect to said exterior surface. The lesion, not shown, is under compression as needle 10 penetrates it. Accordingly, an amount of tissue will enter into slot 18 and be sliced off during distal-to-proximal travel of needle 10, even though cutting edge 20b is recessed with respect to the exterior surface of the needle.

FIG. 7 depicts a fourth embodiment where a slot 18a is formed transversely to the longitudinal axis of needle 10 as in the first embodiment, but the axis of symmetry of slot 18a is normal to the axis of symmetry of slot 18. In other words, the bottom of slot 18a is proximal to the open upper end of slot 18a. Accordingly, a third cutting edge, denoted 20d, is formed. Third cutting edge 20d is auxiliary to first cutting edge 14 in that said third cutting edge slices cellular material from a lesion during proximal-to-distal travel of needle 10.

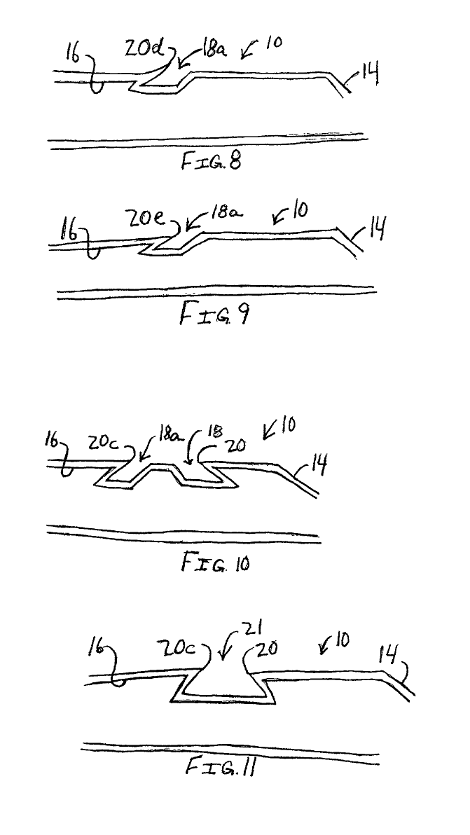

FIGS. 8 and 9 depict fifth and sixth embodiments, respectively, where third cutting edges 20e and 20f are elevated and recessed, respectively, with respect to the exterior surface of needle 10.

A seventh embodiment, depicted in FIG. 10, includes first slot 18 of the first embodiment and second slot 18a of the fourth embodiment. The respective axes of symmetry of said slots are normal to one another and said slots 18 and 18a are longitudinally spaced apart from one another as depicted. In undepicted variations or permutations of this embodiment, second cutting edge 20 is elevated or recessed relative to the exterior surface of needle 10, as is third cutting edge 20d.

FIG. 11 depicts an eighth embodiment. This embodiment, like the seventh, provides two auxiliary cutting edges to augment beveled edge 14. However, it provides a single transversely disposed channel as distinguished from two transversely disposed slots. Thus, instead of slot 18, transversely disposed channel 21 is formed in said needle 10. Channel 21 includes a pair of longitudinally spaced apart undercuts that respectively form second and third cutting edges 20 and 20c. Second cutting edge 20 performs in the same way as second cutting edge 20 of the first embodiment, i.e., it functions during distal-to-proximal travel of needle 10. Third cutting edge 20c, however, functions in the same way as third cutting edge 20c of the fourth embodiment, i.e., during proximal-to-distal travel. Thus, first cutting edge 14 and third cutting edge 20c function during proximal-to-distal travel, but the amount of cellular material sliced from the lesion during such cutting action may not be the same. This eighth embodiment is also similar to the seventh embodiment because second and third cutting edges 20, 20c are flush with the exterior surface of needle 10.

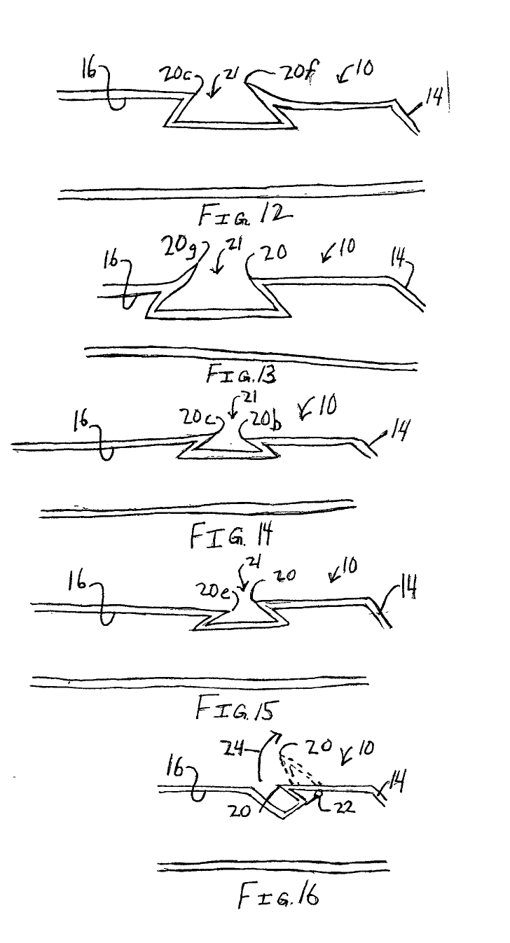

FIGS. 12 and 13 depict ninth and tenth embodiments, respectively, where second cutting edge 20f and third cutting edge 20g are elevated, respectively, with respect to the exterior surface of needle 10.

FIGS. 14 and 15 depict eleventh and twelfth embodiments, respectively, where second cutting edge 20b and third cutting edge 20e are recessed, respectively, with respect to the exterior surface of needle 10.

FIG. 16 provides a longitudinal sectional view where second cutting edge 20 is pivotally mounted by transversely disposed hinge means 22 so that second cutting edge 20 may open as indicated by arcuate directional arrow 24 to the position indicated in dotted lines to cut thicker slices of the suspect lesion during distal-to-proximal travel of the biopsy needle.

Second cutting edge 20 is biased against outward rotation. Various bias means such as different types of springs could be employed but the preferred bias means is a living hinge formed of Nitonel or other suitable material. The hinge is held in its closed, FIG. 16 position when the needle is traveling in a proximal-to-distal direction, i.e., as the needle is inserted, but opens due to frictional forces created by the lesion as depicted in dotted lines in FIG. 16 when the needle is retracted, i.e., when the needle is displaced in a distal-to-proximal direction.

Alternatively, a control means, not depicted, under the control of the physician, causes second cutting edge 20 to pivot about hinge means 22 in small incremental amounts so that the depth of the slice of tissue taken from the lesion is controlled with precision.

It should be understood that, in addition to first cutting edge 20 of the first embodiment, cutting edges 20a, 20b, 20c, 20d, 20e, 20f, and 20g could also be provided in pivotal form and placed under the control of the control means.

Moreover, the cell-collection ability of each cutting edge 20, 20a, 20b, 20c, 20d, 20e, 20f, and 20g may be enhanced by several differing means. For example, empirical studies may show that the cell-cutting ability of said cutting edges is enhanced by making said cutting edges abrasive. A polymer or polymers of the type that cellular material clings to could be added to the cutting edge or edges. Adhesive of the type that cellular material clings to could also be added to said cutting edge or edges.

As drawn, the cut that forms cutting edges 20, 20a, 20b, and 20f are disposed substantially parallel to the bevel cut that forms first cutting edge 14 of needle 10. Accordingly, said cutting edges slice tissue with the same degree of efficiency as first cutting edge 14. The cutting action provided by these cutting edges is during the distal-to-proximal of needle 10 whereas the cutting action provided by first cutting edge 14 is during the proximal-to-distal stroke.

The cut that forms third cutting edges 20c, 20d, 20e, and 20g is substantially normal to the cut that forms second cutting edges 20, 20a, 20b, and 20f. The cutting action of said third cutting edges is during proximal-to-distal motion of the needle and as such said third cutting edges cut cellular material at the same time that first cutting edge 14 cuts cellular material.

This is the first biopsy needle, anywhere in the world, having a dual cutting action. In this way, a physician can reciprocate the needle along its longitudinal axis and obtain multiple slices of tissue for analysis.

Needle 10 is used by inserting it into a lesion in a well-known way and by reciprocating it multiple times along its longitudinal axis. Each proximal-to-distal displacement causes first cutting edge 14 and third cutting edges 20c, 20d, 20e, or 20g, depending upon the embodiment used, to sever part of the lesion and a vacuum pulls such severed parts of the lesion into lumen 16. Each distal-to-proximal motion, however, causes second cutting edges 20, 20a, 20b, or 20f, depending upon the embodiment used, to slice off parts of the lesion. These severed parts of the lesion enter into lumen 16 through slot 18, slot 18a, or channel 21, depending upon the embodiment in use, under the influence of the vacuum. Thus, in a single reciprocation, novel aspiration biopsy needle 10 collects at least twice the amount of lesion material as would a conventional aspiration biopsy needle. The quantity of shaved-off lesion parts collected is increased with each subsequent reciprocation. In this way, a sufficient sample may be taken with a single needle insertion followed by multiple reciprocations.

Additional quantities of lesion material may also be collected by rotating needle 10 about its longitudinal axis of symmetry after a first set of reciprocations has been made. Additional rotations may follow additional reciprocations, it being understood that each rotational adjustment exposes an uncut mass of lesion until the needle has been through three hundred sixty degrees of rotation.

These improvements represent revolutionary improvements in the art of aspiration biopsy needles and this invention is therefore entitled to the status of a pioneering invention. Accordingly, the claims that follow are entitled to broad interpretation, as a matter of law, to protect the heart or essence of the invention.

It will thus be seen that the objects set forth above, and those made apparent from the foregoing description, are efficiently attained. Since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention which, as a matter of language, might be said to fall therebetween.

Now that the invention has been described,

1. An aspiration biopsy needle, comprising:
a needle of elongate, hollow construction having a proximal end and a beveled distal end;
said needle having a uniform diameter along its extent;
said beveled distal end forming a first cutting edge that slices tissue when said needle is inserted into said tissue, said needle being displaced from a proximal position to a distal position during insertion;
a slot formed in said needle near said beveled distal end;
said slot being transversely disposed relative to a longitudinal axis of said needle;
said slot also being angled relative to a transverse axis of said needle such that a bottom of said slot is positioned distal to an opening of said slot;
said opening of said slot being in open communication with an exterior surface of said needle;
said slot including a second cutting edge that slices tissue when said needle is displaced from a distal position to a proximal position; and
means for communicating a vacuum to said proximal end of said needle so that tissue sliced by said first cutting edge during proximal-to-distal travel of said needle is pulled into a lumen of said needle and so that tissue sliced by said second cutting edge during distal-to-proximal travel of said needle is also pulled into said lumen.

2. The needle of claim 1, wherein said slot has a circumferential extent of about one half the circumference of said needle.

3. The needle of claim 1, wherein said second cutting edge is elevated with respect to an exterior surface of said needle.

4. The needle of claim 1, wherein said second cutting edge is recessed with respect to said exterior surface of said needle.

5. The needle of claim 1, further comprising a hinge means to which said second cutting edge is mounted to enable pivotal movement of said second cutting edge.

6. An aspiration biopsy needle, comprising:
a needle of elongate, hollow construction having a proximal end and a beveled distal end;
said needle having a uniform diameter along its extent;
said beveled distal end forming a first cutting edge that slices tissue when said needle is displaced from a proximal position to a distal position;
a first slot formed in said needle near said beveled distal end;
said first slot being transversely disposed relative to a longitudinal axis of said needle;
said first slot also being angled relative to a transverse axis of said needle such that a bottom of said first slot is positioned proximal to an opening of said first slot;
said opening of said first slot being in open communication with an exterior surface of said needle;
said first slot including a second cutting edge that slices tissue when said needle is displaced from a proximal position to a distal position;
means for communicating a vacuum to said proximal end of said needle so that tissue sliced by said first cutting edge during proximal-to-distal travel of said needle is pulled into a lumen of said needle and so that tissue sliced by said second cutting edge during said proximal-to-distal travel of said needle is also pulled into said lumen.

7. The needle of claim 6, wherein said slot has a circumferential extent of about one half the circumference of said needle.

8. The needle of claim 6, wherein said second cutting edge is elevated with respect to an exterior surface of said needle.

9. The needle of claim 6, wherein said second cutting edge is recessed with respect to said exterior surface of said needle.

10. The needle of claim 6, further comprising a hinge means to which said second cutting edge is mounted to enable pivotal movement of said second cutting edge.

11. An aspiration biopsy needle, comprising:
a needle of elongate, hollow construction having a proximal end and a beveled distal end;
said needle having a uniform diameter along its extent;
said beveled distal end forming a first cutting edge that slices tissue when said needle is inserted into said tissue, said needle being displaced from a proximal position to a distal position during insertion;
a first slot formed in said needle near said beveled distal end;
said first slot being transversely disposed relative to a longitudinal axis of said needle;
said first slot also being angled relative to a transverse axis of said needle such that a bottom of said slot is positioned distal to an opening of said slot;
said opening of said first slot being in open communication with an exterior surface of said needle;
said first slot including a second cutting edge that slices tissue when said needle is displaced from a distal position to a proximal position;
a second slot formed in said needle, said second slot being in longitudinally spaced apart relation to said first slot;
said second slot being transversely disposed relative to a longitudinal axis of said needle;
said second slot being angled relative to a transverse axis of said needle such that a bottom of said second slot is positioned proximal to an opening of said second slot;
said opening of said second slot being in open communication with an exterior surface of said needle;
said second slot including a third cutting edge that slices tissue when said needle is displaced from a proximal position to a distal position; and
means for communicating a vacuum to said proximal end of said needle so that tissue sliced by said first and third cutting edges during proximal-to-distal travel of said needle is pulled into a lumen of said needle and so that tissue sliced by said second cutting edge during distal-to-proximal travel of said needle is also pulled into said lumen.

12. The needle of claim 11, wherein said slot has a circumferential extent of about one half the circumference of said needle.

13. The needle of claim 11, wherein said second cutting edge is elevated with respect to an exterior surface of said needle.

14. The needle of claim 11, wherein said second cutting edge is recessed with respect to said exterior surface of said needle.

15. The needle of claim 11, wherein said third cutting edge is elevated with respect to an exterior surface of said needle.

16. The needle of claim 11, wherein said third cutting edge is recessed with respect to said exterior surface of said needle.

17. The needle of claim 11, further comprising a hinge means to which said second cutting edge is mounted to enable pivotal movement of said second cutting edge.

18. The needle of claim 11, further comprising a hinge means to which said third cutting edge is mounted to enable pivotal movement of said third cutting edge.

19. An aspiration biopsy needle, comprising:
a needle of elongate, hollow construction having a proximal end and a beveled distal end;
said needle having a uniform diameter along its extent;
said beveled distal end forming a first cutting edge that slices tissue when said needle is inserted into said tissue, said needle being displaced from a proximal position to a distal position during insertion;
a channel formed in said needle near said beveled distal end;
said channel being transversely disposed relative to a longitudinal axis of said needle;
said channel forming first and second longitudinally spaced apart undercuts in said needle, said first undercut forming a second cutting edge that slices tissue when said needle is displaced in a distal-to-proximal direction and said second undercut forming a third cutting edge that slices tissue when said needle is displaced in a proximal-to-distal direction; and
means for communicating a vacuum to said proximal end of said needle so that tissue sliced by said first and third cutting edges during proximal-to-distal travel of said needle is pulled into a lumen of said needle and so that tissue sliced by said second cutting edge during distal-to-proximal travel of said needle is also pulled into said lumen.

20. The needle of claim 18, wherein said slot has a circumferential extent of about one half the circumference of said needle.

21. The needle of claim 18, wherein said second cutting edge is elevated with respect to an exterior surface of said needle.

22. The needle of claim 18, wherein said second cutting edge is recessed with respect to said exterior surface of said needle.

23. The needle of claim 18, wherein said third cutting edge is elevated with respect to an exterior surface of said needle.

24. The needle of claim 18, wherein said third cutting edge is recessed with respect to said exterior surface of said needle.

25. The needle of claim 18, further comprising a hinge means to which said second cutting edge is mounted to enable pivotal movement of said second cutting edge.

26. The needle of claim 18, further comprising a hinge means to which said third cutting edge is mounted to enable pivotal movement of said second cutting edge.
